(19) United States
(12) Patent Application Publication
(10) Pub. No.: US 2010/0003256 A1
(43) Pub. Date: Jan. 7, 2010

(54) Use of TGF-Beta Antagonists in Treatment of Parathyroid-Related Disorders

(75) Inventors: Chris Lu, Lexington, MA (US); Kelly-Ann Sheppard, Walpole, MA (US); Jeffrey Porter, Lexington, MA (US); Andreas Bauer, Heidelberg (DE)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NOVARTIS AG, Basel (CH)

(21) Appl. No.: 12/315,812

(22) PCT Filed: Jun. 5, 2007
(86) PCT No.: PCT/US2007/013254

§ 371 Date: Dec. 5, 2008

Related U.S. Application Data
(60) Provisional application No. 60/810,890, filed on Jun. 5, 2006.

(51) Int. Cl. A61K 39/395; G01N 33/566; A61K 38/16; A61P 5/18

(52) U.S. Cl. 424/139.1; 435/7.1; 514/12

(57) ABSTRACT

The present invention relates to the use of TGF-β antagonists for the treatment, amelioration, and diagnosis of parathyroid-related disorders, e.g., HPT-JT, familial isolated primary hyperparathyroidism (FIPH), and hyperparathyroidism-jaw tumor (HPT-JT) syndrome, as well as its attendant complications. The present invention also relates to the use of modulators of HRPT2 and its related PAF1 complex for the treatment, amelioration, and diagnosis of TGFβ-related disorders, e.g., pulmonary hypertension, cancer, hypertension, fibrosis, wound healing. Assays of the identification of modulators of HRPT2/PAF1 and SMAD/TGFβ are also provided.

### BACKGROUND OF THE INVENTION

Hyperparathyroidism-jaw tumor (HPT-JT) syndrome is an autosomal dominant disorder characterized by the occurrence of unusual tumors involving the parathyroid gland, facial bones, kidneys, uterus, and possibly, thyroid glands, pancreas and testes. Characteristic lesions include parathyroid adenoma or carcinoma, fibro-osseous lesions of the mandible and maxilla, renal cysts and tumors, and uterine tumors (Chen et al. (2003) J. Intern. Med. 253: 634). The syndrome may be caused by mutations of the tumor suppressor HRPT2 gene, located on chromosome 1q31.2. HRPT2 (hyperparathyroidism-jaw tumor syndrome 2) consists of 17 exons that encode a ubiquitously-expressed 531-amino acid protein, designated parafibromin (named because of its involvement in parathyroid tumors and ossifying fibromas). HRPT2 is the human ortholog of S. cerevisiae (yeast) protein Cdc73, which is present in the yeast PAF1 complex that interacts with RNA polymerase II.

It has been reported that germline HRPT2 mutations are found in 14 of 24 cases of HPT-JT patients and somatic mutations are also detected in all four tumors of sporadic parathyroid carcinomas examined (Carpten et al. (2002) Nat. Genetics 32: 6746; Howell et al. (2003) J. Med. Genetics 40: 657). In addition, HRPT2 mutations were found in 10 of 15 patients with apparently sporadic parathyroid carcinoma (Rubin et al. (2005) J. Clin. Endocrin. Metab. 90(9): 5505). Most HRPT2 mutations are premature truncations that lead to either no expression of the protein or expression of smaller proteins. Indeed, a loss of parafibromin immunoreactivity is a distinguishing feature of parathyroid carcinoma (Tan et al. (2004) Clin Cancer Res. 10(19): 6629).

The TGF-β (transforming growth factor-β) signaling pathway is capable of regulating many cellular activities such as cellular proliferation and differentiation, growth arrest, apoptosis, and epithelial-mesenchymal transdifferentiation (EMT), through regulation of its target genes. Members of the TGF-β family, which include TGF-βs themselves (e.g., TGFβ1, TGFβ2 and TGFβ3), activins and bone morphogenetic proteins (BMPs), are potent regulators of cell proliferation, differentiation, migration and apoptosis. They act through binding to and activating serine/threonine kinase receptors on the cell surface and triggering intracellular signaling pathways in which downstream effector SMAD (vertebrate homologues of MAD, or “Mothers against DPP” proteins) proteins have essential roles (Hata et al. Mol Med Today (1998) 6: 257). SMAD proteins play a key role in intracellular TGF-β signaling and directly translocate to the nucleus upon activation, from where they may activate transcription (Liu et al. (1996) Nature 381:622).

The discovery of methods of modulating HRPT2 or its interactors would be useful for diagnosing, ameliorating the symptoms of, protecting against, and treating HPT-JT, familial isolated primary hyperparathyroidism (FIPH), sporadic parathyroid tumors, and other related malignancies. Likewise, the discovery of methods of modulating TGF-β and its interactors would be useful for diagnosing, ameliorating the symptoms of, protecting against, and treating TGF-β-related disorders and other related malignancies.

### SUMMARY OF THE INVENTION

The present invention provides methods of diagnosing ameliorating the symptoms of, protecting against, and treating parathyroid-related disorders (e.g., HPT-JT, FIPH, sporadic parathyroid tumors, and other related malignancies, etc.), e.g., through use of TGF-β antagonists.

The present invention also provides methods of diagnosing, ameliorating the symptoms of, protecting against, and treating TGF-β-related disorders (e.g., pulmonary hypertension, cancer, hypertension, fibrosis, wound healing), e.g., through use of modulators of HRPT2 and/or the PAF1 complex.

The present invention also provides methods of modulating the signaling TGFβ pathway, e.g., through use of modulators of HRPT2 (or parafibromin) and/or the PAF1 complex. In a preferred embodiment, the method is activation of the TGFβ pathway via antagonizing the HRPT2 gene or its protein product parafibromin.

The present invention also provides methods of identifying and testing agonists and antagonists of TGF-β and its associated proteins, and methods of identifying and testing agonists and antagonists of HRPT2 (or parafibromin), PAF1, and their associated proteins. The present discovery of a protein: protein interaction between parafibromin and SMADs is useful for identifying agents that will enhance or interfere with this binding event (or the resultant complex formation) in vitro or in vivo, and for discovering agents that can be used to treat disorders associated with the absence or presence of this binding event (or the resultant complex formation).

The present invention includes a method of identifying a test agent that modulates (e.g., disrupts) the complex formed between PAF1 complex members and SMADs, comprising: a) providing i) a PAF1 complex member protein or homolog (e.g., parafibromin) capable of binding to a SMAD protein (e.g., SMAD3); ii) a SMAD protein or homolog known to interact with said PAF1 complex member; and iii) one or more test agents for screening; b) mixing, in any order, said PAF1 complex member protein or homolog, said SMAD protein or homolog, and said one or more agents to be tested; and c) measuring the alteration of the PAF1 complex member protein (or analog): SMAD (or analog) binding in the presence of the test agent, as compared to the binding in absence of said agent. The alteration is indicative of the test agent's ability to modulate (e.g., disrupt) the complex formed between PAF1 complex members and SMADs, and the test agent may therefore have therapeutic utility in disorders related to the complex.

In a preferred embodiment, the PAF1 complex member is parafibromin, and the agent is tested for its ability to disrupt the binding of parafibromin and a SMAD protein specifically.

In another preferred embodiment, the SMAD protein is SMAD3, SAMD6, or SMAD7.

The present invention includes a method for screening compounds useful for the treatment of parathyroid-related disorders, comprising contacting a cell expressing parafibromin and a SMAD protein with a compound and detecting a change in the SMAD protein activity and/or TGFβ pathway activity.

The present invention includes a method for screening compounds useful for the treatment of TGFβ-related disorders, comprising contacting a cell expressing parafibromin and a SMAD protein with a compound and detecting a change in the SMAD protein activity and/or TGFβ pathway activity.

This change in SMAD protein activity and/or TGFβ pathway activity may be measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The change in SMAD protein activity and/or TGFβ pathway activity protein may be detected by detecting a change in the interaction between parafibromin (or a protein in the PAF1 complex) and a SMAD protein, by detecting a change in the level of parafibromin, or by detecting a change in the level of one or more of the proteins in the TGFβ pathway. Such cells may be of skeletal muscle origin, may be cultured cells, or may be obtained from or may be within a transgenic organism. Such transgenic organisms include, but are not limited to a mouse, rat, rabbit, sheep, cow or primate.

The present invention is not limited to the native sequence of parafibromin, PAF1, TGFβ, or SMAD polypeptides, or of any other of the polypeptides described. Even where portions or fragments are employed, these portions or fragments may have altered amino acid sequences. For instance, as HPT-JT is caused by mutations of the parafibromin gene, the methods of the present invention, when used with patients suffering from HPT-JT, will pertain to the mutant forms of parafibromin (e.g., which result in truncated versions of the protein parafibromin), and to binding complexes thereof. The present invention also contemplates protein: protein complexes comprising parafibromin or SMAD analogues.

In one embodiment of the above-described method, the parafibromin polypeptide or analog is labeled. In another embodiment, the SMAD potypeptide or analog is labeled.

### BRIEF DESCRIPTION OF THE FIGURES

FIG. 1A shows the results of a coimmunoprecipitation experiment, demonstrating the specific interaction between parafibromin (the protein product of HRPT2) and SMAD6.

FIG. 1B shows a lack of the same interaction between SMAD6 and HRPT2 mutations identified from human HPT-JT patients.

FIG. 2 shows a graphical representation of the effect of HRPT2 or HRPT2 mutant overexpression on TGFβ and BMP-mediated activation of reporter genes.

FIGS. 3A and 3B show a graphical representation of the effect of HRPT2 knockdown using siRNA on TGFβ and BMP mediated-activation of a luciferase reporter gene.

FIGS. 4A-C are graphical representations of the specificity and significance of the PAF1:SMAD interaction. The components of the mammalian PAF1 complex are identified by colored squares. The Y-axis indicates the number of entry points that co-purify with a given protein. SMADs 3, 6 and 7 are the only entry points (out of 335 entry points) that interact with the PAP1 complex (low on the Y-axis). The X-axis represents the significance score. Those interactors that fall furthest to the right are more significant. Both SMAD3 and SMAD7 demonstrate a high degree of significance relative to most proteins that co-purify.

FIG. 5 shows that HRPT2 knockdown sensitizes cells to TGFb stimulated EMT. It is a phase contrast picture depicting that HRPT2 knockdown sensitizes cells to TGF-β-induced invasion and migration

FIG. 6 shows that cells with exposed to the HRPT2 knockdown showed phenotypic change in response to a much lower dose of TGFβ. This figure uses an actin stain.

FIG. 7 shows that cells with exposed to the HRPT2 knockdown showed phenotypic change in response to a much lower dose of TGFβ. This figure uses an E-cadherin stain.

FIG. 8 shows expression level of E-cadherin in these cells at 24 hours and 42 hours.

FIG. 9 depicts the invasion induced by TGFβ, which was increased by about 3-6 fold in cells in which HRPT2 was knocked down by two independent siRNA duplexes.

FIG. 10A shows fluorescence microscope photographs of the samples in FIG. 9. 10B shows that knocking down HRPT2 increases the migration of MDA-MB231 cells in response to TGFβ.

### DETAILED DESCRIPTION OF THE INVENTION

In the present description, the term “treatment” includes both prophylactic or preventive treatment as well as curative or disease suppressive treatment, including treatment of patients predisposed to illness (e.g., to parathyroid-related disorders) as well as ill patients. This term further includes the treatment for the delay of progression of the disease.

By “suppress and/or reverse,” e.g., a parathyroid-related disorder (e.g., a disorder associated with HRPT2 or parafibromin mutations), Applicants mean to abrogate said condition, or to render said condition less severe than before or without the treatment.

“TGF-β antagonists,” as further defined herein, are agents that inhibit TGF-β signal transduction, including TGF-β antagonists which are known in the art. Non-limiting examples include blocking (neutralizing) antibodies specific for a human TGF-β (NAbs), soluble TGF-β receptors, membrane-bound TGF-β receptors, protease inhibitors that inactivate a protease responsible for activating a precursor TGF-β into mature TGF-β, antibodies specific to TGF-β receptors (Types I, II or III) and which prevent TGF-β binding to the receptor, and combinations thereof.

“Cure” as used herein means to lead to the remission of the disorder, e.g., the parathyroid-related disorder, or of ongoing episodes thereof, through treatment.

The terms “prophylaxis” or “prevention” means impeding the onset or recurrence of parathyroid-related disorders, e.g., HPT-JT.

“Delay of progression” as used herein means that the administration of the modulator (e.g., TGFβ antagonist) to patients in a pre-stage or in an early phase of a parathyroid-related disorder in a patient (e.g., HPT-JT) prevents the disease from evolving further, or slows down the evolution of the disease in comparison to the evolution of the disease without administration of the modulator.

“Parathyroid-related disorders,” as used herein, include primary hyperparathyroidism (e.g., parathyroid hyperplasia), familial isolated primary hyperparathyroidism (FIPH), and hyperparathyroidism-jaw tumor (HPT-JT) syndrome, as well as its attendant complications, which include but are not limited to parathyroid adenoma and carcinoma, fibro-osseous lesions of the mandible and maxilla, and uterine tumors. Complications can also include renal cysts and tumors, which are associated with germline HRPT2 mutations. (Zhao et al. (2006) Oncogene, 1.) Non-limiting examples include Wilms tumors and papillary tumors, clear cell carcinomas, oncocytomas, and renal cell carcinomas (e.g., chromophobe RCC). “Parathyroid-related disorders” also include ossifying fibromas, and tumors involving the parathyroid glands (including both sporadic and hereditary parathyroid tumors, of both benign and malignant types), facial bones, kidneys, uterus, thyroid gland, pancreas, and testes. “Parathyroid-related disorders” also include conditions associated with mutations of the HRPT2 gene or parafibromin protein.

“TGFβ-related disorders,” as used herein, includes both disorders related to aberrantly high levels of TGFβ signaling (e.g., hypertension and cancer), and disorders related to aberrantly low levels of TGFβ signaling (e.g., those disorders for which cartilage and/or tendon regeneration is the remedy). A non-limited list of disorders included within the definition of “TGFβ-related disorders” is hypertension (including pulmonary hypertension (e.g., Familial Primary Pulmonary Hypertension, or “FPPH”)), cancer, hypertension, fibrosis, wound healing, Marfan Syndrome, tumor metastasis, congenital heart disease, bone and muscle degenerative diseases, and Arterial Tortuosity Syndrome (ATS) (an autosomal recessive disorder characterized by tortuosity, elongation, stenosis and aneurysm formation in the major arteries owing to disruption of elastic fibers in the medial layer of the arterial wall).

As used herein, “modulate” indicates the ability to control or influence directly or indirectly, and by way of non-limiting examples, can alternatively mean inhibit or stimulate, agonize or antagonize, hinder or promote, and strengthen or weaken.

As used herein a “small organic molecule,” or “small molecule,” is an organic compound (or organic compound complexed with an inorganic compound (e.g., metal) that has a molecular weight of less than 3 kilodaltons, and preferably less than 1.5 kilodaltons.

As used herein a “reporter” gene is used interchangeably with the term “marker gene” and is a nucleic acid that is readily detectable and/or encodes a gene product that is readily detectable such as luciferase.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition/symptom in the host.

“Agent” refers to all materials that may bemused to prepare pharmaceutical and diagnostic compositions, or that may be compounds, nucleic acids, polypeptides, fragments, isoforms, variants, or other materials that may be used independently for such purposes, all in accordance with the present invention.

“Analog” as used herein, refers to a small organic compound, a nucleotide, a protein, or a polypeptide that possesses similar or identical activity or function(s) as the compound, nucleotide, protein or polypeptide or compound having the desired activity and therapeutic effect of the present invention. (e.g., inhibition of tumor growth), but need not necessarily comprise a sequence or structure that is similar or identical to the sequence or structure of the preferred embodiment.

“Derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

“Inhibitors,” or “antagonists” refer to inhibitory molecules—including those identified using in vitro and in vivo assays for TGFβ pathway function—of HRPT2 activity, of TGFβ signaling, or of activity of related proteins (e.g., SMADs, proteins of the PAF1 complex, etc.). Inhibitors and antagonists may be agents that decrease, block, or prevent, signaling via a pathway (e.g., the TGFβ pathway), and/or which prevent the formation of protein complexes such as the PAF1 complex or a histone methyltransferase complex.

As used herein, the term “cancer” includes solid mammalian tumors as well as hematological malignancies. “Solid mammalian tumors” include cancers of the head and neck, lung, mesothelioma, mediastinum, esophagus, stomach, pancreas, hepatobiliary system, small intestine, colon, colorectal, rectum, anus, kidney, urethra, bladder, prostate, urethra, penis, testis, gynecological organs, ovaries, breast, endocrine system, skin, central nervous system including brain; sarcomas of the soft tissue and bone; and melanoma of cutaneous and intraocular origin. The term “hematological malignancies” includes childhood leukemia and lymphomas, Hodgkin's disease, lymphomas of lymphocytic and cutaneous origin, acute and chronic leukemia, plasma cell neoplasm and cancers associated with AIDS. In addition, a cancer at any stage of progression can be treated, such as primary, metastatic, and recurrent cancers. Information regarding numerous types of cancer can be found, e.g., from the American Cancer Society, or from, e.g., Wilson et al. (1991) Harrison's Principles of Internal Medicine, 12th Edition, McGraw-Hill, Inc.

HRPT2

HRPT2 is a tumor suppressor gene located on chromosome 1q31.2 that plays a key role in the regulation of gene expression, particularly in parathyroid tumors (Wang et al. (2005) Horm. Metab. Res. 37:380). The gene consists of 17 exons that encode a ubiquitously-expressed 531-amino acid protein, designated parafibromin (named because of its involvement in parathyroid tumors and ossifying fibromas). Wild-type parafibromin demonstrates an anti-proliferation property, which is likely closely related to the regulation of key cell-cycle related genes. For instance, wild-type parafibromin has been shown to act as a tumor suppressor gene through its inhibition of cyclin D1, which is implicated in parathyroid tumorigenesis (Shattuck et al. (2002) J. Bone. Miner. Res. 17(2): N30). HRPT2 (and parafibromin) mutants lose the ability to inhibit tumorigenetic and cellular proliferative factors, and therefore are thought to engender cancers (particularly of the parathyroid variety).

Comparative studies have shown HRPT2 to be the human ortholog of S. cerevisiae (yeast) protein Cdc73 (GenBank Accession number NP_{—}013522), which is present in the yeast PAF1 complex that consists of PAF1, Cdc73, Leo1, ctr9, and Rtf1. The yeast PAF1 complex has multiple functions, including regulating histone methylation by recruiting a histone methyltransferase, e.g., a set1 complex, to RNA polymerase II, thereby resulting in either transcriptional activation or repression (Rozenblatt-Rosen et al. (2005) Mol. Cell. Biol. 25: 612).

The PAF1 complex is important for histone modification and connections to posttranscriptional events (e.g., histone H3 methylation events), and is implicated in the regulation of genes whose products function in lipid and nucleic acid metabolism, protein synthesis, and cell cycle control (Betz et al. (2002) Mol. Genet. Genomics 268: 272). The mammalian PAF1 complex contains four proteins, PD2, parafibromin, LOC123169, and SH2BP1, which are mammalian homologues of yeast PAF1, Cdc73, Leo1, and ctr9, respectively (Rozenblatt-Rosen et al.).

Hyperparathyroidism-jaw tumor (HPT-JT) syndrome is an autosomal dominant disorder characterized by the occurrence of unusual tumors involving the parathyroid gland, facial bones, kidneys, uterus, and possible thyroid glands, pancreas and testes (Chen et al.), and thought to be caused by mutations of HRPT2. Loss of parafibromin immunoreactivity is a distinguishing feature of parathyroid carcinoma (Tan et al.). Most of HRPT2 mutations are frameshift, non-sense, and splice, which lead to premature truncations associated with either no protein expression or expression of smaller proteins. Some mutant forms of parafibromin fail to associate with the PAF1 complex and the histone methyltransferase complex, further implicating the mutations in cancer.

Incomplete expression of a syndromic form of familial primary hyperparathyroidism (e.g., HPT-JT) may also give, rise to familial isolated primary hyperparathyroidism (FIPH)(Cetani et al (2006) Clin Endocrinol 64(2):146). Furthermore, HRTP2 mutations have also been linked to parathyroid malignancies, including sporadic parathyroid tumors. To date, there is no report yet suggesting how mutations of HRPT2 lead to the development of parathyroid tumors.

TGF-β

The TGF-β (transforming growth factor-β) signaling pathway is capable of regulating many cellular activities such as cellular proliferation and differentiation, growth arrest, apoptosis, and epithelial-mesenchymal transdifferentiation (EMT), through regulation of its target genes. Members of the TGF-β family, which include TGF-βs themselves (e.g., TGFβ1, TGFβ2 and TGFβ3), activins and bone morphogenetic proteins (BMPs), are potent regulators of cell proliferation, differentiation, migration and apoptosis.

TGF-β is a 24 Kd protein produced by many cells, including B and T lymphocytes and activated macrophages, as well as by many other cell types. Among the effects of TGF-β on the immune system are inhibition of IL-2-receptor induction, IL-1-induced thymocyte proliferation, and blocking of gamma interferon-induced macrophage activation. TGF-β is believed to be involved in a variety of pathological conditions (Border et al. (1992) J. Clin. Invest. 90:1), and has been well documented to function as either a tumor suppressor or a tumor promoter.

TGFβ mediates its signaling through two Serine/Threonine kinase cell surface receptors, TGFβRII and ALK5. TGFβ signaling starts with a ligand induced receptor dimerization which allows TGFβII to phosphorylate the ALK5 receptor. The phosphorylation activates ALK5 kinase activity, and the activated ALK5 in turn phosphorylates downstream effector SMAD proteins (vertebrate homologues of MAD, or “Mothers against DPP (decapentaplegic)” proteins), SMAD2 or 3. The, p-SMAD2/3 complexes with SMAD4, enters the nucleus, and activates transcription of the target genes.

SMAD3 is a member of the R-SMAD (receptor-activated SMADs) subgroup of SMADs, and is a direct mediator of transcriptional activation by the TGFβ receptor. TGFβ stimulation leads to phosphorylation and activation of SMAD2 and SMAD3, which form complexes with SMAD4 (a “common SMAD,” or “co-SMAD” in vertebrates) that accumulate with the nucleus and regulate transcription of target genes. R-SMADs localize in the cytoplasm and upon ligand-induced phosphorylation by the TGFβ receptors, form complexes with co-SMAD4 and move to the nucleus, where they regulate gene expression in association with chromatin and cooperating transcription factors. SMAD6 and SMAD7 are inhibitory SMADs (“I-SMADs”), i.e., are transcriptionally induced by TGFβ and function as inhibitors of the TGFβ signaling (Feng et al. (2005) Annu. Rev. Cell. Dev. Biol. 21: 659). SMAD 6/7 exert their inhibiting effects by interfering with the receptor mediated activation of R-SMADs; they associate with type I receptors, competitively interfering with R-SMAD recruitment and phosphorylation. SMAD6 and SMAD7 are known to recruit E3 ubiquitin ligase, which leads to ubiquitination and degradation of SMAD6/7 interacting proteins.

TGF-β has at least two important roles in cancer. It inhibits the growth of many cells, so that loss of responsiveness to TGF-β (e.g., through mutation of receptor or SMAD proteins) results in uncontrolled proliferation. Second, it is highly immunosuppressive, so that tumor cells which no longer respond to TGF-β themselves up-regulate the expression of TGF-β to protect themselves from the immune system. Increased expression of TGF-β may also enhance the ability of the tumor cells to migrate to new sites during metastasis.

TGFβ and HRPT2

As further described in the Experimental section of the present application, tandem affinity purification (TAP) experiments demonstrate a relationship between HRPT2 and the TGFβ signaling pathway, based on detected binding events between the proteins of the PAF1 complex and SMADs 3, 6, and 7. As also described below (and seen in the figures), the relationship is further substantiated by showing an increase in activation of the TGFβ pathway by overexpressing HRPT2 and by knocking HRPT2 down with inhibitory RNAs (siRNA). No effects were seen on TGFβ activation by HRPT2 mutants, which are implicated in disorders such as parathyroid-related disorders (e.g., HPT-JT, FIPH, sporadic parathyroid tumors, and other related malignancies).

In the course of normal physiological events, parafibromin and its complex PAF1 is thought to bind to and form a complex with inhibitory SMADs such as SMAD 6 and 7, thereby suppressing TGFβ activation and signaling. Disruption of this complex results in TGFβ activation due to the loss of the intact complex's inhibitory effects. The disruption of this complex, and therefore, the loss of TGFβ signaling inhibition, can occur due to several events, including (i) presence of HRPT2 mutations (e.g., loss of function mutations, such as those seen in HPT-JT patients); (ii) overexpression of HRPT2, which has the effect of sequestering the other proteins responsible for complex formation away from their native formation; and (iii) knocking down HRPT2 (e.g., by siRNA), which has the effect of preventing complex formation because of lack of HRPT2.

Due to the binding between parafibromin and SMADs, and the resulting effects on TGFβ activation, modulators of HRPT2 (or parafibromin) can be implicated in TGFβ-related disorders. One aim of such modulation is to normalize the parafibromin levels to allow for formation of the complex between, among other things, the proteins of the PAF1 complex and SMADs, for the suppression of TGFβ signaling. Such suppression is useful for treatment of disorders associated with pathologically raised levels of TGFβ signaling (e.g., hypertension and cancer).

The normalization of TGFβ signaling, such as through lowering TGFβ activation levels in disorders characterized by aberrantly high TGFβ activation, is also desired for parathyroid-related disorders, for reasons explained herein. As parathyroid-related disorders are thought to be due to activation of TGFβ as a consequence of overexpression of HRPT2 or knocking down HRPT2, said disorders can be remedied by relieving the abnormally high TGFβ activation, e.g., by administering TGFβ-antagonists.

Conversely, an alternate aim of HRPT2 modulation is to disrupt the complex formed between normal parafibromin proteins, proteins of the PAF1 complex, and SMADs, said disruption leading to TGFβ signaling activation. Such TGFβ activation is useful in disorders associated with low or nonexistent TGFβ signaling. In the case of autoimmune disorders, for example, immunosuppression can be achieved or enhanced through increasing the TGFβ pathway activation (e.g., through disruption of complexes of which parafibromin is a part).

Also due to the observed binding between parafibromin and SMADs, and the resulting effects on TGFβ activation, modulators of TGFβ can be implicated in HRPT2-related disorders (such as HPT-JT, pulmonary hypertension, and ATS). In a fashion analogous to treatment of Marfan Syndrome, a disorder characterized by a mutation that activates TGFβ signaling (and whose effects are remedied by the administration of TGFβ antagonists), the present invention exploits the relationship between HPRT2 mutations and TGFβ signaling (Habashi et al. (2006) Science 312: 117). Habashi describes how mice harboring mutations in the FBN1 gene (encoding fibrillin-1) experience impaired pulmonary alveolar separation associated with increased TGFβ signaling, which can be ameliorated by such TGFβ antagonists as TGFβ neutralizing antibodies (NAb). HRPT2, like FBN1, is a regulator of TGFβ (albeit a regulator whose effects are more of an epigenetic nature than FBN1); therefore, as with Marfan Syndrome, disorders associated with HRPT2 mutations (e.g., pulmonary hypertension and Arterial Tortuosity Syndrome (ATS)) can be treated—and their effects can be mitigated—by the administration of TGFβ antagonists.

TGF-β Antagonists

The effects of TGF-β are mediated by the binding of active TGF-β to specific receptors present on cells, followed by transduction of signal to those cells. TGF-β antagonists are defined as agents that inhibit TGF-β signal transduction, including TGF-β antagonists which are known in the art. For example, agents that bind TGF-β and prevent TGF-β from binding to a TGF-β receptor will act as TGF-β antagonists.

Other non-limiting examples include blocking (neutralizing) antibodies specific for a human TGF-β (NAbs) such as those described by Dasch et al. (J. Immunol. (1989) 142:1536) and Lucas et al. (J. Immunol. (1990) 145:1415), soluble TGF-β receptors, membrane-bound TGF-β receptors, protease inhibitors that inactivate a protease responsible for activating a precursor TGF-β into mature TGF-β, antibodies specific to TGF-β receptors (Types I, II or III) and which prevent TGF-β binding to the receptor, and combinations thereof.

Those skilled in the art recognize various ways in which an antibody derived from one species, for example a mouse, can be engineered in order to be therapeutically useful in a second species, for example a human. Certain of these techniques are briefly reviewed in Harris and Emery, TIBTECH 11:42-44, 1993.

TGF-β is generally secreted as latent precursor consisting of TGF-β non-covalently associated with a protein designated latency-associated protein (LAP; reviewed in Harpel et al. (1992) Prog. Growth Factor Res. 4: 321). This latent complex requires enzymatic cleavage of carbohydrate groups or transient acidification to release the active cytokine. Purified LAP by itself binds active TGF-β with high affinity to form a latent complex. A DNA encoding a 278 amino acid peptide corresponding to pre-pro-TGFβ, terminating just prior to the mature form of TGFβ and containing a Cys33 to Ser33 substitution has been expressed (Derynck et al. (1985) Nature 316: 701), and found to bind TGF-β and render it latent.

Soluble forms of TGF-β receptors will also bind TGF-β and prevent binding to membrane-associated TGF-β receptors. TGF-β receptors are described by Wang et al. (Cell (1991) 67: 797) and Lin et al. (Cell (1992) 68: 775). Soluble forms of TGF-β receptors may be prepared by methods that are known in the art. For example, deletion mutants lacking the transmembrane domain of a TGF-β receptor can be prepared, which will express a soluble TGF-β binding protein. Miyazono et al. (Adv. Immunol. (1994) 55: 181) have reviewed TGF-β receptors

Other types of TGF-β antagonists are also known in the art. For example, Yamaguchi et al. (Nature (1990) 346: 281) discuss decorin, a small chondroitin-dermatan sulphate proteoglycan that binds TGF-β and modulates the activity of this growth factor. Ohtsuki and Massague (Mol. Cell. Biol. 12:261-265, 1992) disclose protein kinase inhibitors that block certain biological activities of TGF-β. T. cruzi produces a cysteine protease (cruzain or cruzipain; Eakin et al. (1992) J. Biol. Chem. 267: 7411) which converts inactive TGF-β precursor into active, mature TGF-β. The design and use of protease inhibitors as drugs is well known in the art (Design of Enzyme Inhibitors as Drugs; Sandier and Smith, eds; 1989, Oxford University Press; Proteinase Inhibitors Medical and Biological Aspects; Katunuma, Umezawa and Holzer, eds., 1983, Springer-Verlag); thus, inhibitors of cruzain van be prepared and will be useful as TGF-β antagonists.

Still other TGF-β antagonists and methods for their production are well known in the art, with many more currently under development. The specific TGF-β antagonist employed is not a limiting feature, as any effective TGF-β antagonist may be useful in the methods of this invention. Examples of such antagonists include monoclonal and polyclonal antibodies directed against one or more isoforms of TGF-β (U.S. Pat. No. 5,571,714 and PCT patent application WO 97/13844), TGF-β receptors, fragments thereof, derivatives thereof and antibodies directed against TGF-β receptors (U.S. Pat. Nos. 5,693,607, 6,008,011, 6,001,969 and 6,010,872 and PCT patent applications WO 92/00330, WO 93/09228, WO 95/10610 and WO 98/48024); latency associated peptide (WO 91/08291), large latent TGF-β (WO 94/09812), fetuin (U.S. Pat. No. 5,821,227), decorin and other proteoglycans such as biglycan, fibromodulin, lumican and endoglin (U.S. Pat. Nos. 5,583,103, 5,654,270, 5,705,609, 5,726,149, 5,824,655, 5,830,847, 6,015,693 and PCT patent applications WO 91/04748, WO 91/10727, WO 93/09800 and WO 94/10187).

Further examples of such antagonists include somatostatin (PCT patent application WO 98/08529), mannose-6-phosphate or mannose-1-phosphate (U.S. Pat. No. 5,520,926), prolactin (PCT patent application WO 97/40848), insulin-like growth factor II (PCT patent application WO 98/17304), IP-10 (PCT patent application WO97/00691), arg-gly-asp containing peptides (U.S. Pat. No. 5,958,411 and PCT patent application WO 93/10808 and), extracts of plants, fungi and bacteria (European patent application 813875, Japanese patent application 8119984 and U.S. Pat. No. 5,693,610), antisense oligonucleotides (U.S. Pat. Nos. 5,683,988, 5,772,995, 5,821,234 and 5,869,462 and PCT patent application WO 94/25588), and a host of other proteins involved in TGF-β signaling, including SMADs and MADs (European patent application EP 874046, PCT patent applications WO 97/31020, WO 97/38729, WO 98/03663, WO 98/07735, WO 98/07849, WO 98/45467, WO 98/53068, WO 98/5,5512, WO 98/56913, WO 98/53830, and WO 99/50296, and U.S. Pat. Nos. 5,834,248, 5,807,708 and 5,948,639) and Ski and Sno (G. Vogel, Science, 286:665 (1999) and Stroschein et al., Science, 286:771-74 (1999)) and fragments and derivatives of any of the above molecules that retain the ability to inhibit the activity of TGF-β.

TGF-β receptors and TGF-β-binding fragments of TGF-β receptors, especially soluble fragments are useful TGF-β antagonists in the methods of the present invention. TGF-β receptors and the nucleic acids encoding them are well known in the art. The nucleic acid sequence encoding TGF-β type 1 receptor is disclosed in GenBank accession number L15436 and in U.S. Pat. No. 5,538,892 of Donahoe et al. The nucleic acid sequence of TGF-β type 2 receptor is publicly available under GenBank accession numbers AW236001; AI35790; AI279872; AI074706; and AA808255. The nucleic acid sequence of TGF-β type 3 receptor is also publicly available under GenBank accession, numbers NM 003243; AI887852; AI817295; and AI681599. In a preferred embodiment, the TGF-β antagonist is an antibody that blocks TGF-β binding to its receptor, or fragments thereof such as F(ab)₂ fragments, Fv fragments, single chain antibodies and other forms of “antibodies” that retain the ability to bind to TGF-β. The antibody may be chimerized or humanized. In this specification, a chimerized antibody comprises the constant region of a human antibody and the variable region of a non-human antibody, such as a murine antibody. A humanized antibody comprises the constant region and framework variable region (i.e. variable region other than the hypervariable region) of a human antibody and the hypervariable region of a non-human antibody, such as a murine antibody. Of course, the antibody can be any other type of antibody derivative, such as a human antibody selected or screened from a phage display system or produced from a xenomouse.

In a more preferred embodiment, the monoclonal antibody is a humanized form of the murine monoclonal antibody 1D11.

In a related embodiment, the TGF-β antagonist is delivered by means of gene therapy, wherein a nucleic acid sequence encoding the antagonist is administered to the patient in vivo or to cells in vitro which are then introduced into a patient, and the antagonist is produced by expression of the produce encoded by the nucleic acid sequence. Methods for gene therapy to deliver TGF-β antagonists are also well known to those of skill in the art. See, for example, PCT patent application WO 96/25178.

Administration of TGF-β Antagonists

The present invention provides methods of using therapeutic compositions comprising an effective amount of a TGF-β antagonist and a suitable diluent and carrier, and methods for treating, e.g., parathyroid-related disorders. The use of TGF-β antagonists in conjunction with soluble cytokine receptors or cytokines, or other immunoregulatory molecules is also contemplated.

For therapeutic use, purified TGF-β antagonist is administered to a patient, preferably a human, for treatment in a manner appropriate to the indication. Thus, for example, TGF-β antagonist compositions administered to augment immune and/or inflammatory function of macrophages can be given by bolus injection, continuous infusion, sustained release from implants, or other suitable technique. Typically, a therapeutic agent will be administered in the form of a composition comprising purified TGF-β antagonist in conjunction with physiologically acceptable carriers, excipients or diluents. Such carriers will be nontoxic to recipients at the dosages and concentrations employed.

Ordinarily, the preparation of such TGF-β antagonist compositions entails combining the TGF-β antagonist with buffers, antioxidants such as ascorbic, acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with conspecific serum albumin are exemplary appropriate diluents. Preferably, product is formulated as a lyophilizate using appropriate excipient solutions (e.g., sucrose) as diluents.

Appropriate dosages can be determined in trials, first in an appropriate animal model, and subsequently in the species to be treated. The amount and frequency of administration will depend, of course, on such factors as the nature and severity of the indication being treated, the desired response, the condition of the individual being treated, and so forth. The appropriate dosages are within the range of about 10 ng/kg/day to about 100 μg/kg/day each or in combination. Preferably a dose of 100 ng/kg/day to about 1000 ng/kg/day for 1-20 days can be expected to induce an appropriate biological effect. Alternatively, bolus injections of from about 1 μg/kg/day to about 100 μg/kg/day can be given at approximately 4-day intervals to exert antimicrobial effects via augmentation of immune and/or inflammatory responses mediated by macrophages/monocytes.

The present invention provides methods of diagnosing, ameliorating the symptoms of, protecting against, and treating parathyroid-related disorders (e.g., HPT-JT, FIPH, sporadic parathyroid tumors, and other related malignancies, etc.), e.g., through use of TGF-β antagonists.

As described herein, aberrant HRPT2 and/or parafibromin levels (e.g., seen in both overexpression and knockdown contexts) have been shown to activate the TGFβ pathway, including enhancing TGFβ target gene expression, enhancing TGFβ-stimulated epithelial-mesenchymal transdifferentiation (EMT) and growth proliferation, and sensitizing the TGFβ RGA response. Therefore, disorders such as parathyroid-related disorders, which are characterized in part by activation of the TGFβ pathway, can be remedied, diagnosed, and ameliorated by antagonizing the TGFβ pathway.

The present invention also provides methods of diagnosing, ameliorating the symptoms of, protecting against, and treating TGF-β-related disorders (e.g., pulmonary hypertension, cancer, hypertension, fibrosis, wound healing), e.g., through use of modulators of parafibromin and/or the PAF1 complex.

That is to say that normalizing the complex formed between parafibromin and/or PAF1 and SMAD proteins can stabilize the TGFβ signaling pathway. Therefore, modulating parafibromin and/or the PAF1 complex can engender treatment of TGFβ-related disorders (i.e., those associated with aberrant TGFβ signaling).

The present invention also provides methods of modulating the signaling TGFβ pathway, e.g., through use of modulators of HPRT2 and/or the PAF1 complex. In a preferred embodiment, the method is activation of the TGFβ pathway via antagonizing the HRPT2 gene or its protein product parafibromin.

The present invention also provides methods of identifying and testing agonists and antagonists of TGF-β and its associated proteins, and methods of identifying and testing agonists and antagonists of HRPT2 (or parafibromin), PAF1, and their associated proteins. The present discovery of a protein: protein interaction between parafibromin and SMADs is useful for identifying agents that will enhance or interfere with this binding event (or the resultant complex formation) in vitro or in vivo, and for discovering agents that can be used to treat disorders associated with the absence or presence of this binding event (or the resultant complex formation).

The present invention includes a method of identifying a test agent that modulates (e.g., disrupts) the complex formed between PAF1 complex members and SMADs, comprising: a) providing i) a PAF1 complex member protein or homolog (e.g., parafibromin) capable of binding to a SMAD protein (e.g., SMAD3); ii) a SMAD protein or homolog known to interact with said PAF1 complex member; and iii) one or more test agents for screening; b) mixing, in any order, said PAF1 complex member protein or homolog, said SMAD protein or homolog, and said one or more agents to be tested; and c) measuring the alteration of the PAF1 complex member protein (or analog): SMAD (or analog) binding in the presence of the test agent, as compared to the binding in absence of said agent. The alteration is indicative of the test agent's ability to modulate (e.g., disrupt) the complex formed between PAF1 complex members and SMADs, and the test agent may therefore have therapeutic utility in disorders related to the complex.

In a preferred embodiment, the PAF1 complex member is parafibromin, and the agent is tested for its ability to disrupt the binding of parafibromin and a SMAD protein specifically.

In another preferred embodiment, the SMAD protein is SMAD3, SAMD6, or SMAD7.

In a preferred embodiment of the invention, cells are created that comprise parafibromin, SMAD proteins, and a TGFβ substrate/reporter construct capable of measuring TGFβ pathway activation. Test agents that may be utilized to modulate the complex formed between PAF1 complex members and SMADs are identified as those that cause TGFβ pathway activation.

Cells useful for expressing parafibromin and SMAD proteins and their associated substrate/reporter constructs include any and all cells that can be maintained in culture and that can be engineered to express a heterologous nucleic acid. The cells may be primary cultures or established cell lines.

The parafibromin, SMAD protein, and TGFβ substrate/reporter construct nucleic acids under the control of suitable transcriptional and translational regulatory sequences can be introduced into the cell by methods known in the art including, for example, transformation, transfection, infection, transduction and injection. The expression vector containing parafibromin, SMAD protein, and TGFβ substrate/reporter construct nucleic acids under the control of suitable promoters is introduced into cells by known methods, for example liposome-mediated transfection, calcium phosphate-mediated transfection, DEAE-dextran transfection, naked DNA transfection, microinjection, electroporation, retroviral-mediated infection, adenoviral-mediated infection, or adeno-associated viral-mediated infection. The reporter construct nucleic acids can be introduced into the cell stably or transiently. Methods for introducing heterologous nucleic acids into eukaryotic cells are described in numerous laboratory manuals including, for example, DNA Cloning: A Practical Approach, vols. I-III (1985) Glover, ed., IRL Press Limited, Oxford, and Sambrook et al. (1989) Molecular Cloning: A Laboratory. Manual, 2.sup.nd edition, Cold Spring Harbor, N.Y.

In a preferred embodiment, the nucleic acid is inserted into a retroviral vector, for example, as described by Pear et al. (1993) Proc. Natl. Acad. Sci. USA 90:8392, incorporated herein by reference. In this embodiment, the viral LTR promoter controls the transcription of the nucleic acid. The vector is transiently transfected into a retroviral packaging line, and the resulting recombinant virus which contains the nucleic acid is harvested, as described by Pear et al., id. The recombinant virus is then, used to infect cells as described by Hoffman et al. (1996) Proc. Natl. Acad. Sci. USA 93:5185, incorporated herein by reference. Cells can be maintained in the undifferentiated state by growing them in tissue culture media containing at least 10% fetal calf serum, or they can be differentiated by growing them in media containing 2% horse serum. The necessary tissue culture methods are known to those of ordinary skill in the art.

In methods for the identification of an agent that modulates (e.g., disrupts) the complex formed between PAF1 complex members and SMADs, the agent may be contacted with the cell comprising parafibromin, SMAD proteins, and the TGFβ substrate/reporter constructs by methods known in the art. For cells in culture or cells obtained from transgenic organisms, the cell may be contacted with the agent by, for example, direct application. The agent may be modified or contained in a delivery vehicle to facilitate entry into the cell. The agent may be isolated and purified, or it may be present in a sample or composition to be subjected to further isolation and purification subsequent to a positive result in the present method. For example, the agent may be contained in a cell lysate, conditioned cell culture media, or a library of synthetic or naturally occurring compounds. For cells present in a transgenic organism, the cells may be contacted with the agent by delivering the agent by methods known in the art, for example by ingestion, parenteral administration, or direct application to tissue surfaces, and may be present in a composition comprising a carrier or diluent. Agents that may be tested in the method of the present invention include, for example, organic and inorganic molecules such as proteins, peptides, lipids, carbohydrates, nucleic acids, including antisense, metals, salts, and so on.

In yet another preferred embodiment, components of the TGFβ pathway are expressed in an in vitro assay, such that agents can be screened to determine their capability of activating TGFβ in vitro. The presence of activated TGFβ substrates can also be assessed using antibodies specific for these proteins.

The present invention includes a method for screening compounds useful for the treatment of parathyroid-related disorders, comprising contacting a cell expressing parafibromin and a SMAD protein with a compound and detecting a change in the SMAD protein activity and/or TGFβ pathway activity.

The present invention includes a method for screening compounds useful for the treatment of TGFβ-related disorders, comprising contacting a cell expressing parafibromin and a SMAD protein with a compound and detecting a change in the SMAD protein activity and/or TGFβ pathway activity.

This change in SMAD protein activity and/or TGFβ pathway activity may be measured by PCR, Taqman PCR, phage display systems, gel electrophoresis, yeast-two hybrid assay, Northern or Western analysis, immunohistochemistry, a conventional scintillation camera, a gamma camera, a rectilinear scanner, a PET scanner, a SPECT scanner, a MRI scanner, a NMR scanner, or an X-ray machine. The change in SMAD protein activity and/or TGFβ pathway activity protein may be detected by detecting a change in the interaction between parafibromin (or a protein in the PAF1 complex) and a SMAD protein, by detecting a change in the level of parafibromin, or by detecting a change in the level of one or more of the proteins in the TGFβ pathway. Such cells may be of skeletal muscle origin, may be cultured cells, or may be obtained from or may be within a transgenic organism. Such transgenic organisms include, but are not limited to a mouse, rat, rabbit, sheep, cow or primate.

The present invention is not limited to the native sequence of parafibromin, PAF1, TGFβ, or SMAD polypeptides, or of any other of the polypeptides described. Even where portions or fragments are employed, these portions or fragments may have altered amino acid sequences. For instance, as HPT-JT is caused by mutations of the HRPT2 gene, the methods of the present invention, when used with patients suffering from HPT-JT, will pertain to the mutant forms of HRPT2 (e.g., which result in truncated versions of the protein parafibromin), and to binding complexes thereof. The present invention also contemplates protein: protein complexes comprising HRPT2 or SMAD analogues.

In one embodiment of the above-described method, the HRPT2 polypeptide or analog is labeled. In another embodiment, the SMAD polypeptide or analog is labeled.

Screening Assays

The invention provides methods (also referred to herein as “screening assays”) for identifying modulators, i.e., candidate or test compounds or agents (e.g., peptides, peptidomimetics, small molecules or other drugs) which bind to parafibromin or SMAD proteins, or to protein members of related complexes (e.g., the PAF1 complex) and have a stimulatory or inhibitory effect on, for example, HRPT2 (or parafibromin) expression or activity, or TGFβ signaling.

The present invention includes a method for compound screening, comprising: a) providing i) a PAF1 complex member protein or homolog (e.g., parafibromin) capable of binding to a SMAD protein (e.g., SMAD3); ii) a SMAD protein or homolog known to interact with said PAF1 complex member; and iii) one or more test compounds for screening; b) mixing, in any order, said PAF1 complex member protein or homolog, said SMAD protein or homolog, and said one or more compound to be tested; and c) measuring the alteration of the PAF1 complex member protein (or analog): SMAD (or analog) binding in the presence of the test compound, as compared to the binding in absence of said compound.

In one embodiment, the invention provides assays for screening candidate or test compounds which bind a parafibromin protein or polypeptide or biologically active portion thereof. In another embodiment, the invention provides assays for screening candidate or test compounds which bind to or modulate the activity of a parafibromin protein or polypeptide or biologically active portion thereof, e.g., modulate the ability of parafibromin to form a PAF1 complex with other proteins normally found in the complex (e.g., PD2, parafibromin, LOC123169, and SH2BP1); and/or modulate the tumor suppressor activity of parafibromin.

In another embodiment, the invention provides assays for screening candidate or test compounds which bind a SMAD (e.g., a SMAD3, SMAD6, or SMAD7) protein or polypeptide or biologically active portion thereof. In another embodiment, the invention provides assays for screening candidate or test compounds which bind to or modulate the activity of a SMAD protein or polypeptide or biologically active portion thereof, e.g., modulate the ability of SMAD6 to inhibit TGFβ and/or to associate with R-SMADs.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al. (1997) Anticancer Drug Des. 12: 145).

Examples of methods for the synthesis of molecular libraries can be found in the art, for example in: DeWitt et al. (1993) Proc. Natl. Acad. Sci. U.S.A. 90: 6909; Erb et al. (1994) Proc. Natl. Acad. Sci. USA 91: 11422; Zuckermann et al. (1994). J. Med. Chem. 37: 2678; Cho et al. (1993) Science 261: 1303; Carrell et al. (1994) Angew. Chem. Int. Ed. Engl. 33: 2059; Carell et al. (1994) Angew. Chem. Int. Ed. Engl. 33: 2061; and in Gallop et al. (1994) J. Med. Chem. 37: 1233.

Libraries of compounds may be presented in solution (e.g., Houghten (1992) Biotechniques 13: 412), or on beads (Lam (1991) Nature 354: 82), chips (Fodor (1993) Nature 364: 555), bacteria (Ladner, U.S. Pat. No. 5,223,409), spores (Ladner '409), plasmids (Cull et al. (1992) Proc Natl Acad Sci USA 89: 1865) or on phage (Scott and Smith (1990) Science 249: 386); (Devlin (1990) Science 249: 404); (Cwirla et al. (1990) Proc. Natl. Acad. Sci. 87: 6378); (Felici (1991) J. Mol. Biol. 222: 301); (Ladner, supra).

In one embodiment, an assay is a cell-based assay comprising contacting a cell expressing HRPT2 (or parafibromin) with a test compound, e.g., a test TGF-β antagonist, and determining the ability of the test compound to modulate (e.g. stimulate or inhibit) the activity of HRPT2. Determining the ability of the test compound to modulate the activity of an HRPT2 can be accomplished, for example, by determining the ability of the TGF-β antagonists to bind to HRPT2, or by determining the ability of the TGF-β antagonists to disrupt the PAF1 complex formation.

In another embodiment, an assay is a cell-based assay comprising contacting a cell expressing a SMAD protein with a test compound and determining the ability of the test compound to modulate (e.g. stimulate or inhibit) the activity of the SMAD protein. Determining the ability of the test compound to modulate the activity of the SMAD protein can be accomplished, for example, by determining the ability of the SMAD protein (e.g., SMAD3, SMAD6, or SMAD7) to bind to parafibromin or by determining the ability of the test compounds to disrupt the SMAD complex formation.

Determining the ability of test TGF-β antagonists to modulate a HRPT2 protein, or of test compounds to modulate SMAD proteins, can be accomplished by determining direct binding. These determinations can be accomplished, for example, by coupling the HRPT2 or SMAD protein with a radioisotope or enzymatic label such that binding of the protein to a test compound (e.g., TGFβ antagonist) can be determined by detecting the labeled protein in a complex. For example, molecules, e.g., proteins, can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

It is also within the scope of this invention to determine the ability of test TGF-β antagonists to modulate a parafibromin protein (or protein members of the PAF1 complex), or to determine the ability of test compounds to modulate SMAD proteins, without the labeling of any of the interactants. For example, a microphysiometer can be used to detect the interaction of test TGF-β antagonists with parafibromin (or with protein members of the PAF1 complex) without the labeling of parafibromin or the test TGF-β antagonists (McConnell et al. (1992) Science 257: 1906). Likewise, the same instrument can be used to detect the interaction of test compounds with SMAD proteins without the labeling of SMAD or the test compounds. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In yet another embodiment, an assay of the present invention is a cell-free assay in which a protein or biologically active portion thereof is contacted with a test compound (e.g., a test TGFβ antagonist, or a compound tested for its ability to modulate SMAD proteins (and, therefore, TGFβ signaling) and the ability of the test compound to bind to the parafibromin protein, or the SMAD protein, or biologically active portions thereof, is determined. Binding of the test compound to the parafibromin protein or the SMAD proteins can be determined either directly or indirectly as described above. In a preferred embodiment, the assay includes contacting the parafibromin protein or biologically active portion thereof with compound known to bind parafibromin to form an assay mixture, contacting the assay mixture with a test compound, and determining the ability of the test compound to interact with an parafibromin protein, wherein determining the ability of the test compound to interact with an parafibromin protein comprises determining the ability of the test compound to preferentially bind to parafibromin in or biologically active portion thereof as compared to the known compound.

In another preferred embodiment, the assay includes contacting SMAD proteins (e.g., SMAD6) or biologically active portion thereof with compound known to bind SMAD to form an assay mixture, contacting the assay mixture with a test compound, and determining the ability of the test compound to interact with a SMAD protein, wherein determining the ability of the test compound to interact with an SMAD protein comprises determining the ability of the test compound to preferentially bind to SMAD or biologically active portion thereof as compared to the known compound.

Such a determination may be accomplished using a technology such as real-time Biomolecular Interaction Analysis (BIA). Sjolanderet al., 1991 Anal. Chem. 63:2338-2345 and Szabo et al., 1995 Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize parafibromin or SMAD to facilitate separation of complexed from uncomplexed forms of the protein, as well as to accommodate automation of the assay. Binding of a test compound to parafibromin or SMAD can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, glutathione-S-transferase/kinase fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and the non-adsorbed parafibromin or SMAD protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of binding determined using standard techniques.

Other techniques for immobilizing proteins on matrices can also be used in the screening assays of the invention. For example, parafibromin or SMAD can be immobilized utilizing conjugation of biotin and streptavidin. Biotinylated parafibromin or SMAD protein or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with parafibromin or SMAD proteins or target molecules can be derivatized to the wells of the plate, and unbound parafibromin or SMAD protein trapped in the wells by antibody conjugation. Methods for detecting such complexes, in addition to those described above for the GST-immobilized complexes, include immunddetection of complexes using antibodies reactive with the parafibromin or SMAD protein or target molecules.

In yet another aspect of the invention, the parafibromin or SMAD proteins can be used as “bait proteins” in a two-hybrid assay or three-hybrid assay (see, e.g., U.S. Pat. No. 5,283,317; Zervos et al., 1993 Cell 72:223-232; Madura et al., 1993 J. Biol. Chem. 268:12046-12054; Bartel et al., 1993 Biotechniques 14:920-924; Iwvabuchi et al., 1993 Oncogene 8:1693-1696; and Brent WO94/10300), to identify other proteins which bind to parafibromin or SMAD. Such parafibromin- or SMAD-binding proteins are also likely to be involved in the propagation of signals by the parafibromin or SMAD proteins.

The two-hybrid system is based on the modular nature of most transcription factors, which consist of separable DNA-binding and activation domains. Briefly, the assay utilizes two different DNA constructs. In one construct, the gene that codes for an parafibromin or SMAD protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a kinase dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the parafibromin or SMAD protein which interacts with the protein.

This invention further pertains to novel agents identified by the above-described screening assays. Accordingly, it is within the scope of this invention to further use an agent identified as described herein in an appropriate animal model. For example, an agent identified as described herein (e.g., an HRPT2 or SMAD modulating agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The following examples are merely illustrative and not meant to limit the scope of the present claims in any manner.

### Examples

### Example 1

### Association Between TGFβ and parafibromin/PAF1

In order to identify novel modulators of TGFβ pathway, we applied a systematic tandem affinity purification (TAP) method to several known signaling molecules of TGFβ pathway (including using SMADs 1,2, 3, 4, 5, 6, 7, and 8; ALK3, 5, and 6; TGFβII; BMPRII; Smurf1,2; and TAK1 as bait proteins). As described in Rigaut et al. (Nat Biotechnol. (1999) 17(10): 1030), the contents of which are hereby incorporated by reference, the TAP purification method involves the fusion of the TAP tag to the target protein of interest and the introduction of the construct into the cognate host cell or organism.

The TAP tag is a tandem fusion of (i) IgG-binding units of protein A from Staphylococcus aureus (ProtA); and (ii) the Calmodulin Binding Peptide (CBP), separated by a TEV protease cleavage site. It allows the rapid purification of complexes from a relatively small number of cells without prior knowledge of the complex composition, activity, or function. Combined with mass spectrometry, the TAP strategy allows for the identification of proteins interacting with a given target protein

Tandem Affinity Purification and Mass Spectrometry.

Retroviral transduction vectors were generated by cloning open reading frames, amplified by polymerase chain reaction, into a Moloney-based vector with the Gateway site-specific recombination system (Life Technologies). Virus stocks were generated in a HEK293 Gag-Pol packaging cell line. HEK293T cells were infected and complexes were purified by using a modified TAP protocol1, which were transiently transfected. The proper localization of all TAP-tagged proteins was monitored by indirect immunofluorescence. Cells were grown in DMEM medium with 10% FCS. For stimulation the medium was replaced with either fresh medium containing 2 ng/ml TGFβ1, or 50 ng/ml BMP2 or fresh medium alone. Cells were stimulated for 2 hours for SMAD3, 4 hours for SMAD6 and SMAD7. Cells were harvested by mechanical detachment, washed with excess PBS on ice and lysed in immunoprecipitation buffer. Protein samples were separated by SDS-PAGE, complete gel lanes were systematically cut into slices and proteins were digested in-gel with trypsin as described in Shevchenko (Shevchenko, A., Wilm, M., Vorm, O. & Mann, M. Mass spectrometric sequencing of proteins silver-stained polyacrylamide gels. Anal. Chem. 68, 850-858 (1996). Protein identification was performed by LCM-MS/MS, and MS data were searched against an in-house curated version of the International Protein Index (IPI), maintained at the EBI (Hinxton, UK). Results of database searches were read into a database system for further bioinformatics analysis.

Data Analysis

A significance score is given to each candidate identification by the database that is reflecting the statistical significance of the finding in a defined set of experiments. The significance score weights specificity, reproducibility and the quality of the identification (e.g. identification by the mass spectrometer). Significant candidates are selected from the list and represented separately in a network graph.

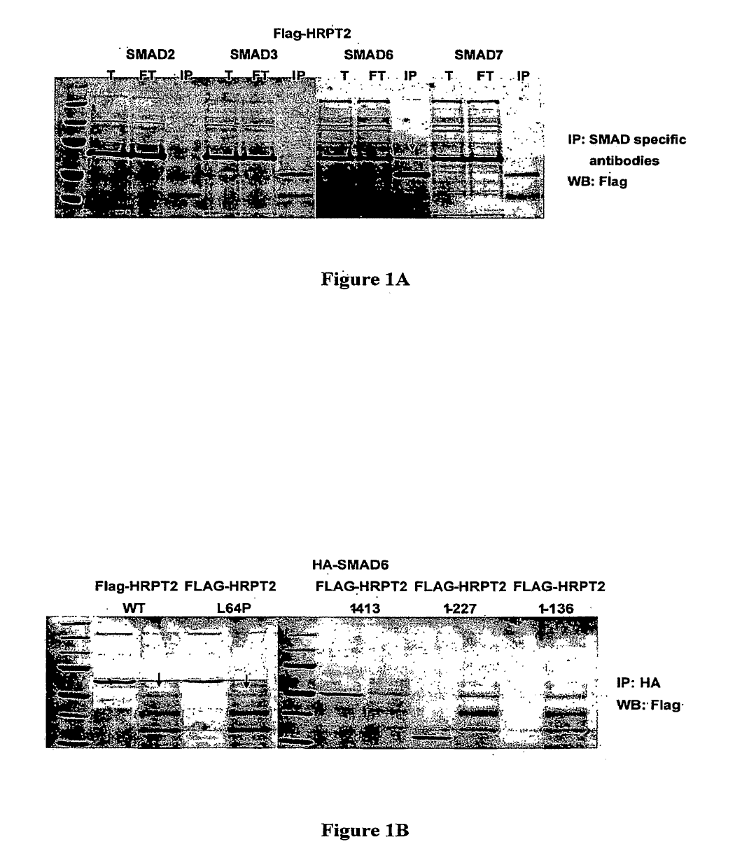

All four members of the PAF1 complex, HRPT2, PD2, LOC123169 and SH2BP1, were found to be exclusively associated with SMAD3 and SMAD6 or SMAD7. As seen in FIGS. 1A and 1B, confirmation by immuno-coprecipitation (Co-IP) shows that HRPT2 interacts specifically with SMAD6, whereas three HRPT2 mutants found in human HPT-JT patients fail to do so, suggesting that this specific HRPT2 and SMAD6 interaction may be important in HPT-JT pathogenesis.

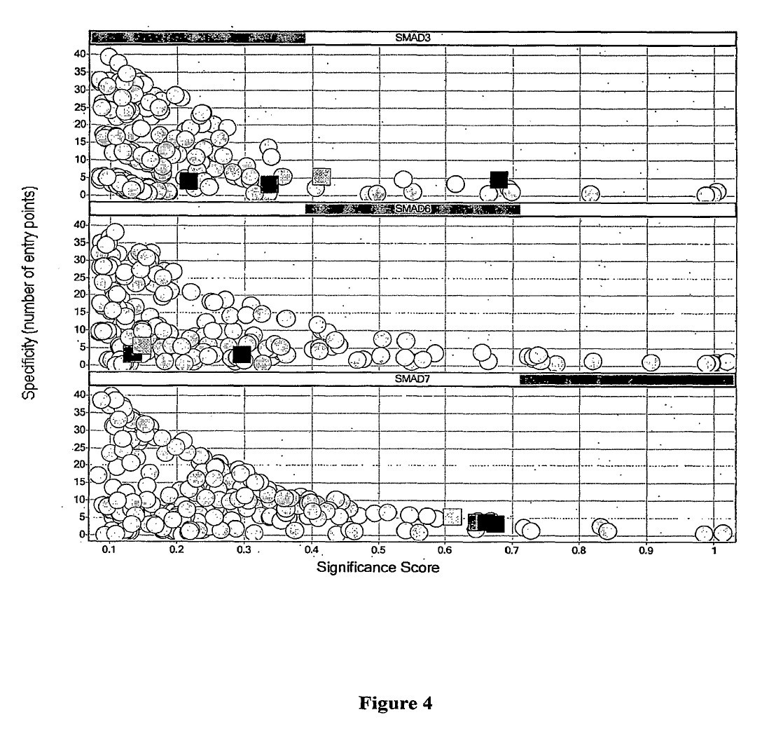

FIG. 4 demonstrates the specificity and significance of the PAF1:SMAD interaction. The components of the mammalian PAF1 complex are identified by colored squares. The Y-axis indicates the number of entry points that co-purify with a given protein. SMADs 3, 6 and 7 are the only entry points (out of 335 entry points) that interact with the PAF1 complex (low on the Y-axis). Only one other entry point has been shown to interact with one component of the PAF1 complex in a reproducible manner. The X-axis represents the significance score. Those interactors that fall furthest to the right are more significant. Both SMAD3 and SMAD7 demonstrate a high degree of significance relative to most proteins that co-purify.

### Example 2

### Correlation Between HRPT2 Expression and TGFβ Activation

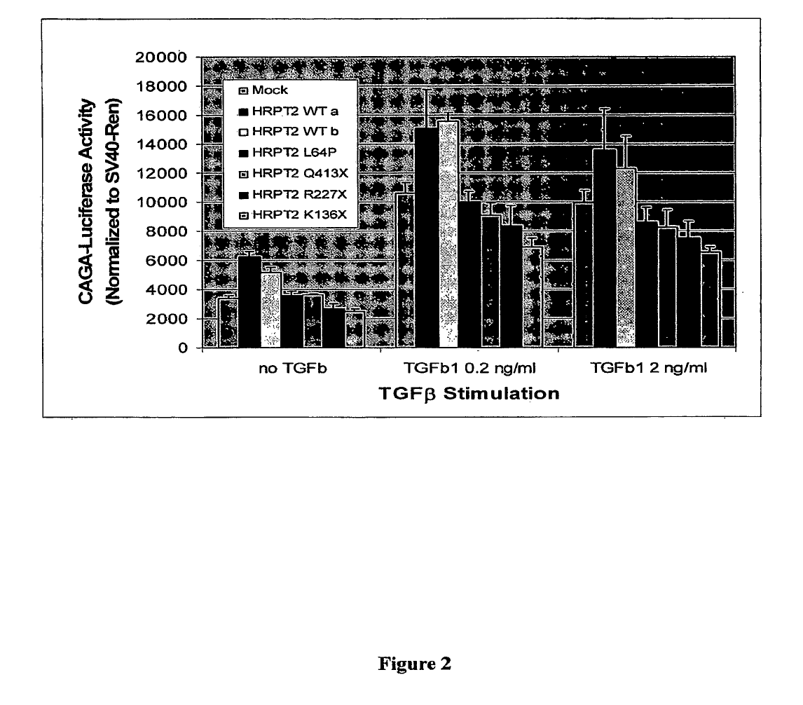

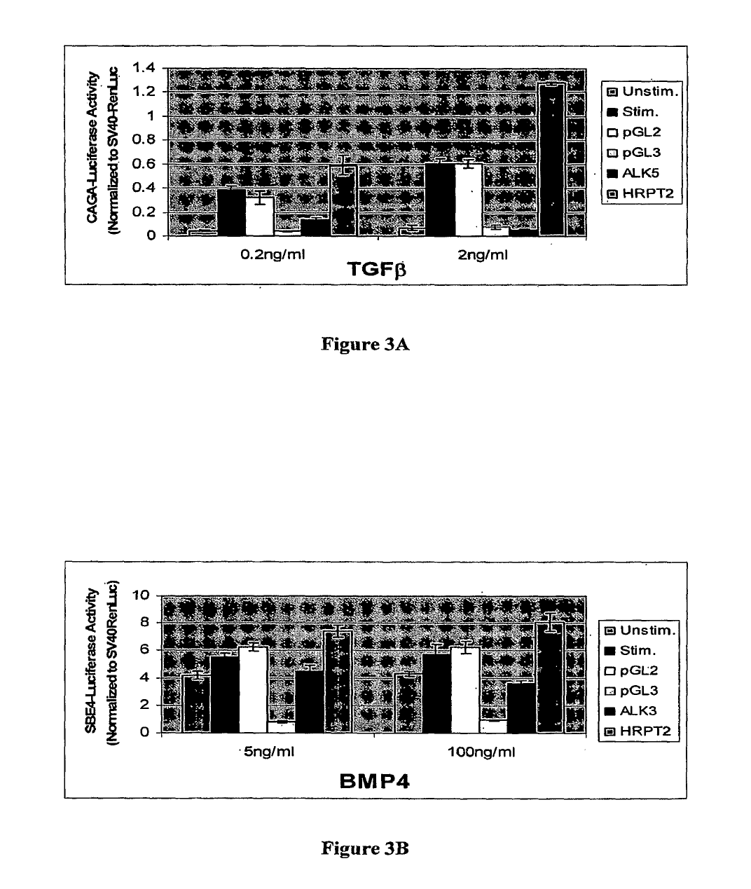

As seen in FIG. 2, overexpression of HRPT2 cDNA leads to activation of TGFβ pathway in a luciferase reporter assay, and again, overexpression of the HRPT2 mutants has no effect. In addition, knockdown of HRPT2 expression with siRNA also leads to increased TGFβ signaling (FIGS. 3A and 3B). Loss of HRPT2 heterozygosity is expected to have the same effect as knocking down HRPT2 expression, i.e., enhancing TGFβ signaling activation. Enhancing TGFβ signaling activation leads to epithelial-mesenchymal transdifferentiation (EMT), cellular proliferation, and ultimately, tumor formation.

The cDNA encoding the full-length of HRPT2 or HRPT2 mutants were cloned into the expression vector pcDNA3.1. The resulting expression constructs were co-transfected with a TGFb responsive firefly luciferase reporter and a SV40 promoter driven renella luciferase plasmids pCAGA-Luc into HEK293T cells. Thirty-six hours post-transfection the cells were stimulated with TGFb (2 ng/ml) for 18-24 hours. Luciferase activity was assayed using the Promega Dual-Glo Kit from according to the manufacturers protocol. The firefly luciferase activity was normalized using the renella luciferase activity as a transfection efficiency control.

HEK293T/17 cells were transfected with 50 nM of commercially-available siRNA duplexes (Dharmacon HRPT2 SMARTpool reagent (cat. #015184)) using the Mirus Trans1T-LT1 TKO reagent (cat. #MIR 2300) in 10% FBS. Twenty-four hours post-transfection, cells were then transfected with the CAGA 12-Luc. promoter reporter and SV-40-Luc. Renilla control using the Roche FuGENE 6 transfection Reagent (cat. #1 815 091). Twenty-four hours post plasmid transfection, cells were stimulated with TGFb. Luciferase activity was measured using the Promega Dual-Glo kit (cat. #E2980).

### Example 3

### Characterization of HRPT2 in TGFβ Regulated Cellular Activities

The following assays are used to further confirm the ability of the HRPT2/PAF1 complex to regulate TGFβ activities.

Endogenous target gene expression may be performed through the use of quantitative PCR on TGF-β pathway members, such as PAI-1, SnoN, P21, SMAD6 and SMAD7, as follows: TGFb is well documented to stimulate transcription of many target genes including the above described. HRPT2 gene expression can be knocked down by siRNA in HaCaT cells, and the effects of knockdown on TGFb stimulated transcription of these genes can be measured by quantitative PCR.

Through the performance of a phospho-SMAD2/3 assay, TGFβ stimulation should lead to a rapid phosphorylation of SMAD2 and SMAD3 in 10-60 minutes. This assay demonstrates the ability of HRPT2 to enhance TGFβ stimulated p-SMAD2/3 kinetics, and as described above, the effect of HRPT2 siRNA knockdown can be assessed using the phosphor-SMAD assays.

Phenotypic responses of these assays directly correlate with cancer inhibition. The effects of siRNA knockdown of HRPT2 in HaCaT cells on TGFβ induced growth arrest, apoptosis, and EMT can be measured the same assay system described above

### Example 4

### Cellular Confirmation of the Interaction Between PAF1 Complex and SMAD

Histone methyltransferase (HMT) assay: The PAF1 complex has been shown to associate with histone methytransferase complexes. SMAD6 or SMAD7 can be immunoprecipitated using an anti-SMAD6 or -SMAD7 antibody, respectively, and the immunoprecipitants assayed for HMT activity using a Kit from Upstate (catalog #17-330).

Chromatin Immunoprecipitation (ChIP) assay: As described in greater detail in Strahl-Bolsinger et al. (Genes Dev. (1997) 11: 83-93), the contents of which are hereby incorporated by reference, an anti-HRPT2 antibody can be used for ChIP and to check if the CAGA-Luc reporter and/or PAI, SMAD7, and SnoN promoters are occupied by the HRPT2/PAF1 complex.

### Example 5

### Pathological Analysis of TGFβ Signaling and Parathyroid Cancer

Tissue samples obtained from both normal subjects, and those with parathyroid tumors can be examined for SMAD2 or SMAD3 phosphorylation levels, and for TGFβ target gene (e.g. PAI-1, SMAD7 and SnoN) expression levels.

Explant cultures are conducted on the tissue samples, which are then stimulated with TGFβ for comparison between the normal and diseased, i.e., to see if there is an elevated TGFβ response in the parathyroid tumor samples compared to the normal samples (using phosphor-SMAD2 assay, qPCR assay on PAI-1, SnoN and SN4AD7, and proliferation/apoptosis assay). The ability of TGFβ receptor I kinase inhibitors to reverse the effects of the elevated TGFβ response supports claims of TGFβ antagonists utility in the treatment of parathyroid tumors.

### Example 6

### HRPT2 Knockdown Sensitizes NMuMG Cells to TGFβ Induced Epithelial-Mesenchymal Transition (EMT)

Epithelial-mesenchymal transition (EMT) is a fundamental mechanism extant during development. In healthy adults, EMT is a physiological response for appropriate regeneration upon tissue/cell injury; when misregulated it can lead to such diseases as fibrosis and tumorigenesis. This cellular transformation involves a loss of cell adhesion and apical-basal polarity, followed by a shift in cytoskeleion dynamics toward the mesenchyinal phenotype. The phenotypic change is also accompanied with formation of actin stress fibers and delocalization and down regulation of E-cadherin at the cell junctions. NMuMG cells, a mammary gland epithelial derived cell line, shows a typical and apparent phenotypic change reflecting EMT in response to TGFβ.

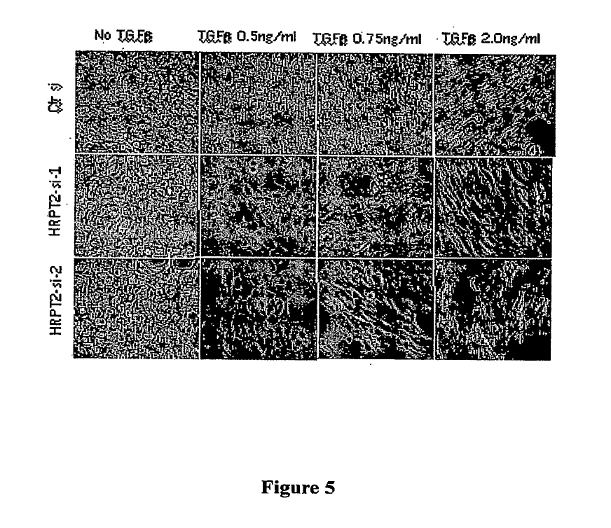

NMuMG cells were treated with two individual HRPT2 siRNA duplexes, as well as a non-specific siRNA as control. Two days after transfection, the cells were treated with various doses of TGFβ for 24 hours, and then fixed and stained for actin and E-Cadherin. FIG. 5 is a phase contrast picture depicting that HRPT2 knockdown sensitizes cells to TGF-β-induced invasion and migration.

In the absence of TGF-β, NMuMG cells showed a typical epithelial cell phenotype. After 24 h of TGFβ treatment, this regular, cobblestone-like pattern became a spindle-shaped fibroblast-like pattern. In the control cell line treated with a non-specific siRNA, cells underwent a phonotypical change at 2 ng/ml of TGFβ, while in cells in which HRPT2 was knocked down, the cells demonstrated a phenotypic change at much lower dose, around 0.75 ng/ml of TGFβ.

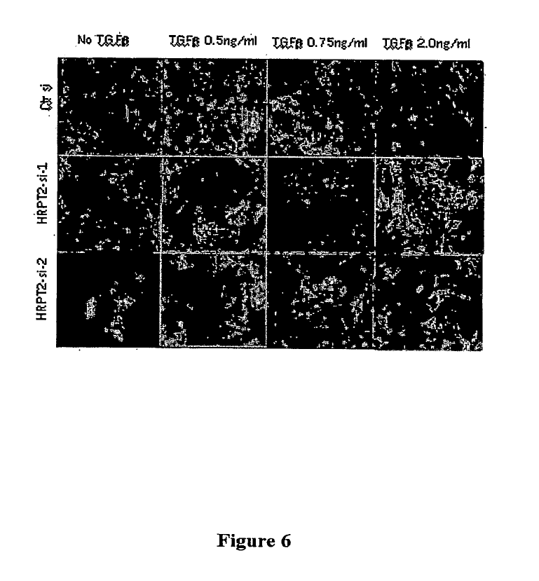

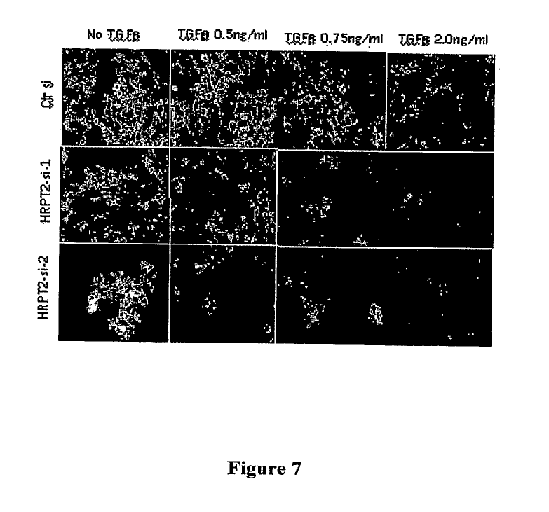

FIGS. 6 and 7 show actin and E-cadherin staining, respectively. In cells treated with TGFβ, formation of actin stress fibers, and delocalization and down-regulation of E-cadherin at the cell junctions, were observed. Similar to FIG. 5, cells with exposed to the HRPT2 knockdown showed phenotypic change in response to a much lower dose of TGFβ.

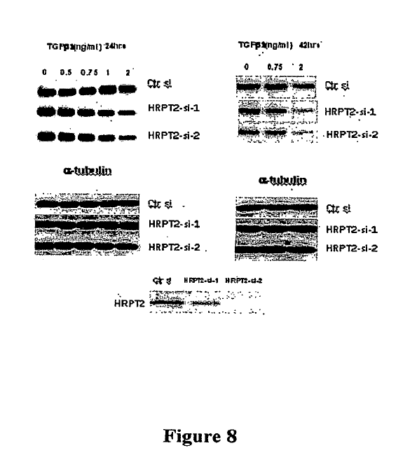

Expression level of E-cadherin in these cells was detected by Western blot. FIG. 8 shows the expression level of E-cadherin in these cells at 24 hours and 42 hours. As expected, E-cadherin expression in cells with HRPT2 knockdown was reduced in response to lower TGFβ stimulation compared to control cells at both time points, consistent with phenotypic changes described herein. These data indicate that knockdown HRPT2 sensitized NMuMG cells to TGFβ induced EMT. Given that EMT is one of the major mechanisms of tumorigenesis, HRPT2 is thought to exert its tumor suppressor function by interacting with Smad3 and inhibit TGFβ-induced EMT.

### Example 7

### HRPT2 Knockdown Sensitizes NMuMG Cells to TGFβ Induced Invasion and Migration

One important step after cells undergo through EMT is increased migration and invasion, either for developing cells to move to a new location and differentiate, or for tumor cells to relocate to a more nutritious environment. As described above, knocking down HRPT2 sensitizes NMuMG cells to TGFβ induced EMT. In view of TGFβ's supposed role in inducing the invasive phenotype in many epithelial cancers, knocking down HRPT2 was investigated for its effect on cellular invasion as induced by TGFβ.

NMuMG cells were transfected with HRPT2 siRNA and control siRNA as previously described, and then the cells were split onto three dimensional matrigel chamber wells and cultured in the presence or absence of TGFβ. After one day of treatment, cells that progressed through the gel were stained and counted; multiple wells were counted for each sample and the average number was calculated.

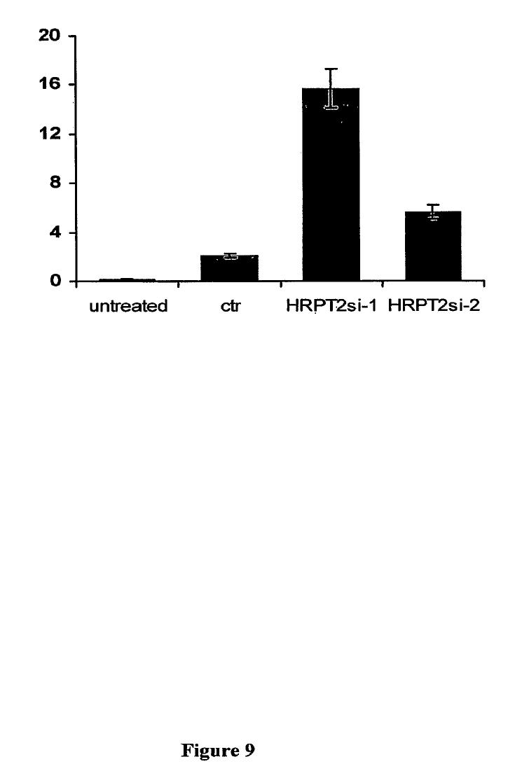

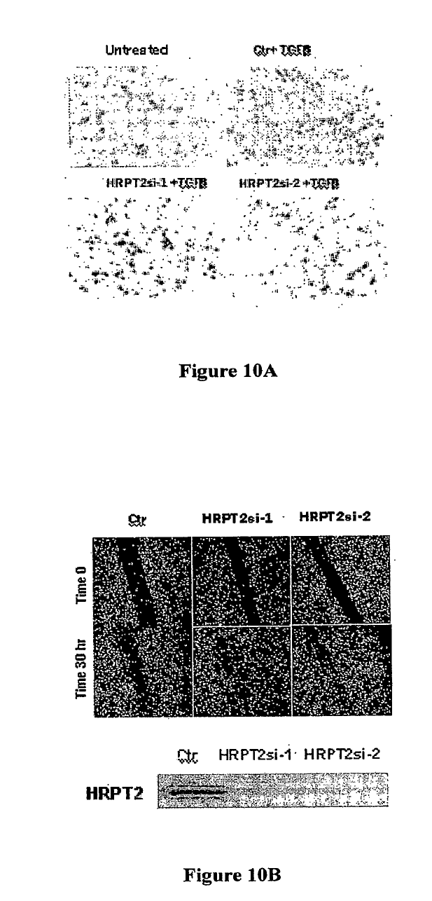

As FIG. 9 shows, the invasion induced by TGFβ was increased by about 3-6 fold in cells in which HRPT2 was knocked down by two independent siRNA duplexes. FIG. 10A shows fluorescence microscope photographs of the samples. This data indicates that knocking down HRPT2 also sensitizes NMuMG cells to TGFβ induced invasion.

The HRPT2 knockdown effect on migration in MDA-MB231 cells, a malignant and highly migratory human breast cancer cell line, was also tested. As similarly shown in FIG. 5, knocking down HRPT2 increases the migration of MDA-MB231 cells in response to TGFβ (see FIG. 10B). Collectively, the sensitization of both NMuMG cells and MDA-MB231 cells to TGFβ induced invasion and migration when engendered by knocking down HRPT2, indicates that loss of HRPT2 function contributes to tumorigenesis (e.g., by allowing the cells migrate and invade through intercellular matrix), and hence survival.

These experiments support HRPT's function as a tumor suppressor partly by inhibiting TGFβ's pro-oncogenic effects. Mutations of HRPT2 sensitize cells to TGFβ-induced EMT, migration, invasion, and can ultimately lead to cancers.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

We claim:

1. A method of treating or preventing a parathyroid-related disorder in a patient, comprising administering to the patient a TGFβ antagonist.

2. The method of claim 1, wherein said TGFβ antagonist is a polyclonal neutralizing antibody (NAb).

3. The method of claim 1, wherein said TGFβ antagonist is a soluble TGF-β receptor.

4. The method of claim 1, wherein said TGFβ antagonist is a small molecule.

5. The method of claim 1, wherein said TGFβ antagonist is a TGFβ receptor kinase inhibitor.

6. The method of claim 1, wherein the parathyroid-related disorder is HPT-JT.

7. The method of claim 1, wherein the parathyroid-related disorder is primary hyperparathyroidism (e.g., parathyroid hyperplasia).

8. The method of claim 1, wherein the parathyroid-related disorder is familial isolated primary hyperparathyroidism (FIPH).

9. The method of any of claims 2-5, wherein the parathyroid-related disorder is HPT-JT.

10. A method of treating or preventing a TGFβ-related disorder in a patient, comprising administering to the patient an agent capable of normalizing the binding event between the PAF1 complex and SMAD proteins.

11. The method of claim 10, further comprising administering to the patient an agent capable of normalizing HRPT2 expression levels or parafibromin protein levels.

12. The present invention includes a method of identifying a test agent that modulates the complex formed between PAF1 complex members and SMADs, comprising:
a) providing a PAF1 complex protein or homolog capable of binding to a SMAD protein, a SMAD protein or homolog known to interact with said PAF1 complex protein or homolog, and a test agent for screening;
b) mixing, in any order, said PAF1 complex protein or homolog, said SMAD protein or homolog, and said test agent for screening PAF1 complex protein or homolog, said SMAD protein or homolog, and said agent to be tested; and
c) measuring the alteration of the binding between said PAF1 complex protein or homolog and said SMAD protein or homolog in the presence of the test compound, as compared to the binding in absence of said compound.

13. The method of claim 12, wherein the PAF1 complex protein is parafibromin.

14. The method of claim 12, wherein the SMAD protein is any one of SMAD3, SMAD6, and SMAD7.

15. A method for screening compounds useful for the treatment of parathyroid-related disorders, comprising contacting a cell expressing parafibromin and a SMAD protein with a compound and detecting a change in the SMAD protein activity and/or TGFβ pathway activity.

16. A method for screening compounds useful for the treatment of TGFβ-related disorders, comprising contacting a cell expressing parafibromin and a SMAD protein with a compound and detecting a change in the SMAD protein activity and/or TGFβ pathway activity.

17. A method of determining whether a patient is suffering from or at risk for a parathyroid-related disorder, the method comprising: the patient;
b) contacting said test sample with a SMAD protein; and
c) determining whether parafibromin protein within said sample is capable of binding to said SMAD in the same fashion as parafibromin contained within a comparable biological sample obtained from normal tissue, a difference indicating a parafibromin mutation and resultant presence of or risk for a parathyroid disorder.

18. The method of claim 17 wherein the SMAD is any one of SMAD3, SMAD6, and SMAD7.
